(19) United States
(12) Patent Application Publication
(10) Pub. No.: US 2023/0348955 A1
(43) Pub. Date: Nov. 2, 2023

(54) METHODS OF PRODUCING TARGET CAPTURE NUCLEIC ACIDS

(71) Applicant: The Regents of the University of California, Oakland, CA (US)

(72) Inventors: Richard Green, Santa Cruz, CA (US); Balaji Sundararaman, Santa Cruz, CA (US)

(21) Appl. No.: 17/783,927

(22) PCT Filed: Dec. 18, 2020
(86) PCT No.: PCT/US2020/065972

§ 371 Date: Jun. 9, 2022

Related U.S. Application Data
(60) Provisional application No. 62/950,720, filed on Dec. 19, 2019.

(51) Int. Cl. C12Q 1/6811

(52) U.S. Cl. CPC C12Q 1/6811

(57) ABSTRACT

Provided are methods of producing target capture nucleic acids. The methods comprise bidirectionally amplifying a circular nucleic acid template by rolling circle amplification (RCA) using first and second primers, where the circular nucleic acid template comprises a target nucleotide sequence and a restriction site. The bidirectional amplification produces a double-stranded concatemer comprising a first strand comprising a plurality of linked units, each unit comprising the target nucleotide sequence and the restriction site, and a second strand which is the reverse complement of the first strand. The methods further comprise digesting the double-stranded concatemer using a restriction endonuclease that cleaves the restriction site to produce a plurality of restriction fragments. Also provided are target capture nucleic acids produced according to such methods. Methods of capturing target nucleic acids using target capture nucleic acids produced according to such methods are also provided.

### CROSS-REFERENCE TO RELATED APPLICATIONS

This application claims the benefit of U.S. Provisional Patent Application No. 62/950,720, filed Dec. 19, 2019, which application is incorporated herein by reference in its entirety.

### STATEMENT OF GOVERNMENT SUPPORT

This invention was made with Government support under contract MG-30-17-0045-17 awarded by Institute for Museum and Library Services. The Government has certain rights in the invention.

### INTRODUCTION

High coverage nucleic acid sequencing is necessary in a variety of contexts, including the discovery and validation of rare mutations for cancer diagnostics. However, cost prohibits high coverage sequencing of the whole genome. Targeted sequencing of regions of interest instead of the whole genome is used to identify rare variants. Sequencing of the gene(s) frequently mutated in cancer is widely used to discover driver mutations. Target gene-specific drugs are effective only in patients with specific driver mutations. Targeted sequencing of select transcripts is also used in personalized medicine. Companion diagnostic methods sequence selective genes at high coverage, whose mutations and expression levels indicate the effectiveness of personalized therapies.

Targeted sequencing of selected polymorphic sites in the genome is used in forensic sciences, e.g., for the identification of the source of rare and low amount DNA specimens recovered from the crime sites. Targeted sequencing has also been applied for analyzing ancient DNA samples recovered from paleontological and archaeological sites. Forensic and ancient DNA samples are highly prone to contamination by unwanted DNA and contain very low amounts of DNA of interest. Non-targeted sequencing is wasteful for these samples and data are difficult to interpret due to contamination. Hence, the enrichment of genomic DNA of interest has been attempted, but the methods are laborious and expensive. An inexpensive method to enrich whole genomic DNA is needed for the analysis of a wide range of species in research, clinical, forensic and paleogenomic contexts.

State of the art bait synthesis for targeted sequencing involves solid phase oligonucleotide synthesis or in vitro transcription. Both methods have drawbacks. First, incomplete chemical synthesis of the ends of long oligos results in variations of the probe sequence. Second, large scale synthesis is expensive and reagent replenishment requires significant turnaround time. Third, RNA baits have stability issues that limit their long term storage required in clinical diagnostic laboratories.

### SUMMARY

Provided are methods of producing target capture nucleic acids. The methods comprise bidirectionally amplifying a circular nucleic acid template by rolling circle amplification (RCA) using first and second primers, where the circular nucleic acid template comprises a target nucleotide sequence and a restriction site. The bidirectional amplification produces a double-stranded concatemer comprising a first strand comprising a plurality of linked units, each unit comprising the target nucleotide sequence and the restriction site, and a second strand which is the reverse complement of the first strand. The methods further comprise digesting the double-stranded concatemer using a restriction endonuclease that cleaves the restriction site to produce a plurality of restriction fragments, each restriction fragment comprising a target capture nucleic acid comprising the reverse complement of the target nucleotide sequence. Also provided are target capture nucleic acids produced according to such methods.

Methods of capturing target nucleic acids are also provided. Such methods comprise combining target capture nucleic acids produced according to the methods of the present disclosure and a sample comprising a target nucleic acid. The combining is under conditions in which a target capture nucleic acid of the target capture nucleic acids specifically hybridizes to the target nucleic acid to produce a target capture nucleic acid-target nucleic acid complex. Such methods further comprise isolating the target capture nucleic acid-target nucleic acid complex.

### BRIEF DESCRIPTION OF THE FIGURES

FIG. 1: Schematic illustration of target capture nucleic acid (sometimes referred to herein as “probe”) synthesis according to one embodiment of the present disclosure. In this example, a target sequence oligonucleotide with 5′ and 3′ flanking sequences is employed. A splint adapter hybridizes with the head-to-tail of the target oligonucleotide. The splint adapter mediates head-to-tail intramolecular ligation of the target oligonucleotide. Forward and reverse primers bind in between the restriction enzyme (RE) site and poly-dA/dT site. RCA initiated by the forward primer and the newly synthesized product serves as the template for the reverse primer. Restriction enzyme digestion of the RCA product results in target capture probes having the target sequence. Non-limiting examples of sequences that may be employed are indicated by the sequence identifiers in the dashed boxes.

FIG. 2: Schematic illustration of target capture nucleic acid (or “probe”) synthesis for whole genome enrichment according to one embodiment of the present disclosure. In this example, target genomic DNA (gDNA) is fragmented and size selected for 100-200 bp fragments. gDNA fragments are ligated with a bridge oligonucleotide at the 3′ end facilitated by a splint adapter. Bridge-ligated gDNA fragments are head-to-tail ligated using another splint adapter generating a circular product. Forward and reverse primers bind between the RE site and poly-dA/dT site. RCA initiated by the forward primer and the newly synthesized product serves as the template for the reverse primer. Restriction enzyme digestion of RCA product results in capture probes with the target sequence. Non-limiting examples of sequences that may be employed are indicated by the sequence identifiers in the dashed boxes.

FIG. 3: Capillary gel electrophoresis of RCA products. RCA amplifications were performed using Phi29 DNA polymerase with forward and reverse primers for 30 minutes (R0.5), 2 hr (R2), 4 hr (R4), 8 hr (R8) and 24 hr (R24). RCA products were resolved on a Fragment Analyzer instrument using the High Sensitivity Genomic (50 kb) kit. DNA traces indicate that RCA produced high molecular weight DNA products.

FIG. 4: Capillary gel electrophoresis of RCA products. RCA products digested by restriction enzyme after annealing complementary oligos to the RE site resulted in near complete digestion of RCA products to produce monomeric target capture probes ˜80 nucleotides in length.

FIG. 5: Mitochondrial read coverage shown as circular plot. The outermost line of the circular plot shows the mitochondrial DNA (mtDNA) coordinates, and the innermost circle is the histogram of read coverage for pre-capture library. Read coverage histograms for HVR1 and HVR2 target enrichment are shown in the inner circular plots.

FIG. 6: Scatter plots of average coverage of SNPs in autosomes and sex chromosomes distinguish male and female samples. X chromosomal SNPs have twice the average coverage in female samples compared to male samples as shown in Panel A. And panel B shows that female samples have no coverage in Y chromosomal SNPs.

FIG. 7: Coverage of Horse SNPs compared against probe length and GC content. Panel A shows that 80 bp long probes have 2-fold higher coverage than 50 bp and 100 bp probes. SNP coverage is higher for probes with 40-70% GC content with a peak coverage around 55% as shown in Panel B.

### DETAILED DESCRIPTION

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Producing Target Capture Nucleic Acids

Provided are methods of producing target capture nucleic acids. The methods comprise bidirectionally amplifying a circular nucleic acid template by rolling circle amplification (RCA) using first and second primers, where the circular nucleic acid template comprises a target nucleotide sequence and a restriction site. The bidirectional amplification produces a double-stranded concatemer comprising a first strand (which may also be referred to herein as a “first concatemer”) comprising a plurality of linked units, each unit comprising the target nucleotide sequence and the restriction site, and a second strand (which may also be referred to herein as a “second concatemer”) which is the reverse complement of the first strand. The methods further comprise digesting the double-stranded concatemer using a restriction endonuclease that cleaves the restriction site to produce a plurality of restriction fragments, each restriction fragment comprising a target capture nucleic acid comprising the reverse complement of the target nucleotide sequence.

The methods of the present disclosure find use in a variety of applications, including but not limited to targeted sequencing of DNA and/or RNA in various research, clinical, forensic, and paleogenomic applications. For example, current approaches for synthesizing probes for targeting nucleic acid sequencing, which include solid phase oligonucleotide synthesis and in vitro transcription, have substantial drawbacks. First, incomplete chemical synthesis of the ends of long oligos results in variations of the probe sequence. In addition, large scale synthesis is expensive and reagent replenishment requires significant turnaround time. Moreover, RNA probes have stability issues that limit their long-term storage required, e.g., in clinical diagnostic labs. The methods of the present disclosure overcome these drawbacks by providing an inexpensive and rapid isothermal amplification approach to probe synthesis. In addition, the present methods do not require large scale synthesis of oligonucleotides because the templates are amplified by RCA. The RCA reaction can produce microgram quantities of probes in less time and at significantly less expense as compared to the current chemical synthesis approaches. Details regarding embodiments of the present methods will now be provided.

As used herein, a “target capture nucleic acid” is a nucleic acid strand that comprises the reverse complement of a target nucleotide sequence. The target nucleotide sequence is the sequence of a target nucleic acid or portion thereof. Because the target capture nucleic acid comprises the reverse complement of the target nucleotide sequence, the target capture nucleic acid may be used to capture the target nucleic acid or portion thereof present in a sample of interest. Captured target nucleic acids may then be isolated and subjected to downstream analysis, e.g., targeted nucleic acid sequencing, or the like.

The length of the target capture nucleic acid may vary and depend, e.g., upon the nature of the target nucleic acid or portion thereof. In certain embodiments, the length of the target capture nucleic acid is from 10 nucleotides (nt) to 500 nt. For example, the target capture nucleic acid may be at least 10 nt in length, but 500 nt or less, 450 nt or less, 400 nt or less, 350 nt or less, 300 nt or less, 275 nt or less, 250 nt or less, 225 nt or less, 200 nt or less, 175 nt or less, 150 nt or less, 125 nt or less, 100 nt or less, 75 nt or less, 50 nt or less, or 25 nt or less in length. According to some embodiments, the target capture nucleic acid is 500 nt or less in length, but 10 nt or greater, 25 nt or greater, 50 nt or greater, 75 nt or greater, 100 nt or greater, 125 nt or greater, 150 nt or greater, 175 nt or greater, 200 nt or greater, 225 nt or greater, 250 nt or greater, 275 nt or greater, 300 nt or greater, 350 nt or greater, 400 nt or greater, or 450 nt or greater in length. The portion of the target capture nucleic acid that is the reverse complement of the target nucleotide sequence may be 50% or greater, 55% or greater, 60% or greater, 65% or greater, 70% or greater, 75% or greater, 80% or greater, 85% or greater, 90% or greater, 95% or greater, or 99% or greater of the total length of the target capture nucleic acid, e.g., a target nucleic acid having any of the lengths provided above.

The present methods comprise bidirectionally amplifying a circular nucleic acid template by rolling circle amplification (RCA) using first and second primers. As used herein, the term “rolling circle amplification” or “RCA” refers to an amplification (e.g., isothermal amplification) that generates linear concatemerized copies of a circular nucleic acid template using a strand-displacing polymerase. During RCA, the polymerase continuously adds single nucleotides to a primer (e.g., an oligonucleotide primer or a primer produced by nicking a double-stranded circular DNA (e.g., using an endonuclease)) annealed to the circular template which results in a concatemeric single-stranded DNA (ssDNA) that contains tandem repeats (or “linked units”) (e.g., tens, hundreds, thousands, or more tandem repeats) complementary to the circular template. Suitable strand-displacing polymerases that may be employed include, but are not limited to, Phi29 polymerase, Bst polymerase, Vent exo-DNA polymerase, and the like. Reagents, protocols and kits for performing RCA are known and include, e.g., the RCA DNA Amplification Kit available from Molecular Cloning Laboratories; and TruePrime™ RCA Kit available from Expedeon.

As used herein, an “oligonucleotide” is a single-stranded multimer of nucleotides from 5 to 500 nucleotides, e.g., 5 to 100 nucleotides. Oligonucleotides may be synthetic or may be made enzymatically, and, in some embodiments, are 5 to 50 nucleotides in length. Oligonucleotides may contain ribonucleotide monomers (i.e., may be oligoribonucleotides or “RNA oligonucleotides”), deoxyribonucleotide monomers (i.e., may be oligodeoxyribonucleotides or “DNA oligonucleotides”), or a combination thereof. Oligonucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. In some embodiments, the template oligonucleotide comprise one or multiple target sequences.

According to the present methods, the amplification is bidirectional because first and second primers are employed, where the first primer is complementary to the circular template and initiates the RCA reaction, and where the second primer is complementary to the newly synthesized RCA product and initiates linear amplification in the opposite direction. Hence, the isothermal amplification is bidirectional and generates both sense and antisense strands of the target nucleotide sequence. In certain embodiments, the first primer, the second primer, or both, comprise a sequence that hybridizes to the restriction site.

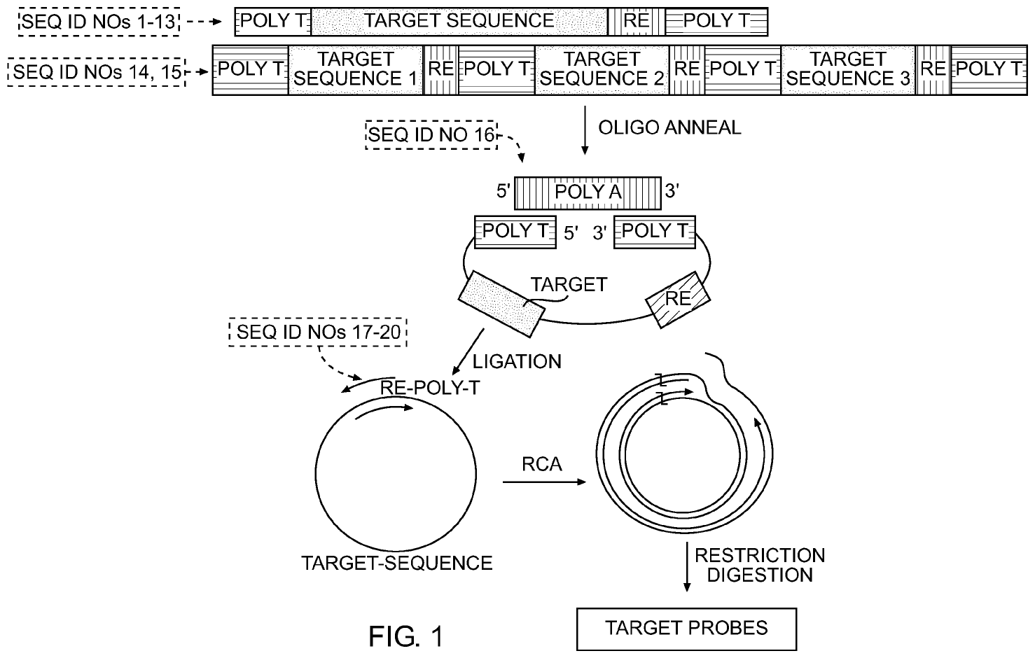

An example approach for producing target capture nucleic acids according to one embodiment is schematically illustrated in FIG. 1.

In some embodiments, prior to the bidirectional amplification, the methods further comprise producing the circular nucleic acid template by circularizing a linear nucleic acid comprising the target nucleotide sequence and the restriction site. Circularizing a linear nucleic acid may be performed using any suitable approach. In one example, the two ends of the linear nucleic acid are ligated to each other using a suitable ligase, e.g., a ligase suitable for blunt end ligation or sticky end ligation. Blunt end ligation could be employed by providing a blunt end at one end of the linear nucleic acid and a blunt end at the other end of the linear nucleic acid. Sticky end ligation could be employed by providing a sticky end at one end of the linear nucleic acid and a complementary sticky end at the other end of the linear nucleic acid.

According to some embodiments, circularizing the linear nucleic acid is achieved by splint ligation. For example, the circularized DNA may be produced from a linear nucleic acid that includes a first sequence at a first end and a second sequence at the end opposite the first end, where circularization is achieved using a splint oligonucleotide that includes sequences complementary to the first and second sequences. In certain embodiments, the linear nucleic acid comprises a poly dT domain at each of its ends, where the splint ligation comprises hybridizing a poly dA splint oligonucleotide to the poly dT domains, and where the circular nucleic acid template comprises a poly dA/poly dT site resulting from the splint ligation. In certain embodiments, the first primer, the second primer, or both, comprise a sequence that hybridizes to at least a portion of the poly dA/poly dT site. According to some embodiments, a Gibson assembly approach or modified version thereof (e.g., NEBuilder Hifi DNA assembly) is used to join the ends of the linear nucleic acid using a splint oligonucleotide.

In certain embodiments, when the methods further comprise producing the circular nucleic acid template by circularizing a linear nucleic acid by splint ligation, the linear nucleic acid is stabilized for splint ligation using a single-strand stabilizing protein. According to some embodiments, the single-strand stabilizing protein is single-stranded nucleic acid binding protein (SSB). SSB binds in a cooperative manner to single-stranded nucleic acid (ssNA) and does not bind well to double-stranded nucleic acid (dsNA). Upon binding ssNA, SSB destabilizes helical duplexes. SSBs that may be employed include prokaryotic SSB (e.g., bacterial or archaeal SSB) and eukaryotic SSB. Non-limiting examples of SSBs that may be employed include E. coli SSB, E. coli RecA, Extreme Thermostable Single-Stranded DNA Binding Protein (ET SSB), Thermus thermophilus (Tth) RecA, T4 Gene 32 Protein, replication protein A (RPA—a eukaryotic SSB), and the like. ET SSB, Tth RecA, E. coli RecA, T4 Gene 32 Protein, as well buffers and detailed protocols for preparing SSB-bound ssNA using such SSBs are available from, e.g., New England Biolabs, Inc. (Ipswich, MA). Suitable protocols for stabilizing ssNA with SSBs are available and typically included in kits comprising SSBs.

Subsequent to the circularization reaction and prior to RCA of the circular template, the circularization reaction mixture may be treated with a nuclease that only degrades linear DNA to remove any remaining (uncircularized) linear nucleic acid prior to RCA.

In certain embodiments, when the methods further comprise circularizing a linear nucleic acid to produce the circular nucleic acid template, the methods further comprise producing the linear nucleic acid prior to its circularization. According to some embodiments, producing the linear nucleic acid comprises attaching a nucleic acid comprising the restriction site to a nucleic acid comprising the target nucleotide sequence. Any suitable approach for attaching the nucleic acids may be employed. In certain embodiments, the attaching is by splint ligation. For example, the nucleic acid comprising the restriction site may include a first sequence at an end and the nucleic acid comprising the target nucleotide sequence may include a second sequence at an end, where the attaching is achieved using a splint oligonucleotide that includes sequences complementary to the first and second sequences.

According to some embodiments, the linear nucleic acid comprises a genomic DNA fragment. A non-limiting example of such a genomic DNA fragment is a bacterial artificial chromosome (BAC) DNA fragment. In certain embodiments, the linear nucleic acid comprises a genomic DNA fragment and producing the linear nucleic acid comprises fragmenting genomic DNA to produce genomic DNA fragments, size-selecting the genomic DNA fragments, where the size-selected genomic DNA fragments comprise the genomic DNA fragment, and attaching a nucleic acid comprising the restriction site to the genomic DNA fragment. According to some embodiments, the size-selected genomic DNA fragments are from 50 to 300 nt in length, e.g., from 100 to 200 nt in length.

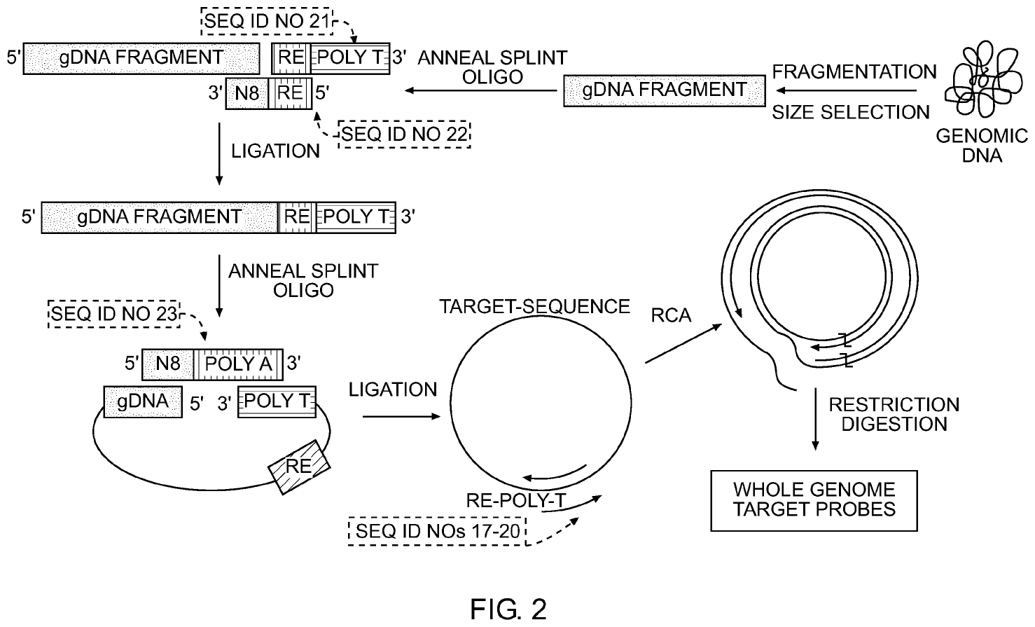

An example approach for producing a linear nucleic acid comprising a genomic DNA fragment and a restriction site is schematically illustrated in FIG. 2. Starting at the top right, genomic DNA (gDNA) is fragmented and size selected to produce size-selected gDNA fragments. A splint oligonucleotide containing random nucleotides (“N8”) (SEQ ID NO: 22) is then annealed to an end of a size selected gDNA fragment and an end of a “bridge” nucleic acid comprising the restriction site (“RE”) and the poly-dT region (SEQ ID NO: 21), followed by ligation to produce the linear nucleic acid comprising the size selected genomic DNA fragment and the bridge nucleic acid containing a restriction site and a poly-dT/dA region. A second oligonucleotide complementary to the bridge nucleic acid in one end and comprising random nucleotides in the other end (SEQ ID NO: 23) is annealed to the gDNA fragments with RE site and poly-dT/dA region by head-to-tail fashion, followed by ligation to produce circularized gDNA fragments that is bridged by a RE site and poly-dT/dA region. In certain embodiments, the first and second splint ligation may be done in the same reaction as one step ligation comprising the size selected gDNA fragments, bridge nucleic acid and the two splint oligonucleotides. In certain embodiments, the first and second splint oligos combined into one single oligonucleotide (SEQ ID NO:25), annealed with the bridge oligo (SEQ ID NO: 24) and genomic fragments are circularized with bridge oligo in one step splint ligation. Also shown in FIG. 2 is the circularization of the linear nucleic acid via splint ligation, followed by bidirectional RCA amplification and restriction digestion to produce the target capture nucleic acids (“whole genome target probes”).

The circular nucleic acid template comprises a restriction site. A “restriction site” refers to a nucleotide sequence recognized and cleaved by a given restriction endonuclease. In certain embodiments, the restriction site present in the circular nucleic acid template is for a restriction endonuclease that generates cohesive (or “sticky”) ends, including but not limited to, AscI, AvaI, BamHI, BclI, BglII, BstEI, Bst, BI, BstYI, EcoRI, MluI, NarI, NheI, NotI, PstI, PvuI, SacI, SalI, SpeI, StyI, XbaI, XhoI and XmaI. According to some embodiments, the restriction site present in the circular nucleic acid template is for a restriction endonuclease that generates blunt ends, including but not limited to, EcoRV, FspI, NaeI, NruI, PvulI, SmaI, SnaBI, and StuI.

In some embodiments, the randomers in the splint oligonucleotides (SEQ ID NOs:21, 23, 25) are from 3 nt to 31 nt in length, e.g., 6 nt, 8 nt, 10 nt in length. These random nucleotides synthesized by randomly incorporating four conventional nucleotides (A, T, G, C) generate a multitude of combination of sequences. The numbers of different sequence combinations formed by the randomers depend on the length of the oligonucleotides, for example a 10 nt randomer nucleic acid will comprise 4-10 combinations to form 1,048,576 different sequences. These diverse sequence combinations form complementary sequences to the ends of genomic DNA fragments to facilitate splint ligation.

In some embodiments, the conditions of the RCA (e.g., temperature, duration, polymerase employed, and/or the like) are such that the double-stranded concatemer comprises 500 or more, 750 or more, 1000 or more, 5000 or more, 10,000 or more, 50,000 or more, 100,000 or more, 200,000 or more, 300,000 or more, 400,000 or more, 500,000 or more, 600,000 or more, 700,000 or more, 800,000 or more, 900,000 or more, or 1,000,000 or more of the linked units.

As will be appreciated, the nature of the double-stranded concatemer and, in turn, the target capture nucleic acids, will vary depending upon the nucleotides employed during the bidirectional amplification. The term “nucleotide” is intended to include those moieties that contain not only the naturally occurring purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain haptens, binding members, labels (e.g., fluorescent labels) and/or the like, and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

Accordingly, in certain embodiments, the target capture nucleic acids are deoxyribonucleic acids (DNAs). According to some embodiments, the target capture nucleic acids are ribonucleic acids (RNAs). In certain embodiments, the target capture nucleic acids comprise both deoxyribonucleotides and ribonucleotides. According to some embodiments, the target capture nucleic acids comprise modified nucleotides incorporated into the double-stranded concatemer during the bidirectional amplification. A variety of useful modified nucleotides may be incorporated during the bidirectional amplification, non-limiting examples of which include binding member-labeled nucleotides, thermostability-increasing nucleotides, and/or the like. In certain embodiments, when the target capture nucleic acids comprise binding member-labeled nucleotides incorporated into the double-stranded concatemer during the bidirectional amplification, the binding member-labeled nucleotides comprise biotin-labeled nucleotides. Such binding member-labeled nucleotides find use, e.g., for isolating target nucleic acids from a nucleic acid sample using, e.g., beads or other types of solid supports that comprise surfaces that bind to the binding member-labeled nucleotides, e.g., streptavidin coated beads for immobilizing and isolating target capture nucleic acid-target nucleic acid complexes where the target capture nucleic acid comprise biotin-labeled nucleotides. According to some embodiments, when the target capture nucleic acids comprise thermostability-increasing nucleotides incorporated into the double-stranded concatemer during the bidirectional amplification, the thermostability-increasing nucleotides comprise 2-Amino-2′-deoxyadenosine-5′-Triphosphate (2-Amino-dATP), 5-Methyl-2′-deoxycytidine-5′-Triphosphate (5-Me-dCTP), 5-Propynyl-2′-deoxycytidine-5′-Triphosphate (5-Pr-dCTP), 5-Propynyl-2′-deoxyuridine-5′-Triphosphate (5-Pr-dUTP) and or halogenated deoxy-uridine (XdU) like 5-Chloro-2′-deoxyuridine-5′-Triphosphate (5-Cl-dUTP), 5-Bromo-2′-deoxyuridine-5′-Triphosphate (5-Br-dUTP), or any combination thereof.

The target nucleotide sequence will vary depending upon the nature of the target nucleic acid to be captured using the target capture nucleic acids. Non-limiting examples of a target nucleotide sequence include a target genomic DNA sequence, a target cell-free DNA (cfDNA) sequence, a target circulating tumor DNA (ctDNA) sequence, a target ribonucleic acid (RNA) sequence, or a target complementary DNA (cDNA) sequence.

Aspects of the present disclosure further include target capture nucleic acids produced according to any of the methods of producing target capture nucleic acids of the present disclosure.

### Methods of Capturing Target Nucleic Acids

Aspects of the present disclosure further include methods of capturing target nucleic acids. In certain embodiments, such methods comprise combining target capture nucleic acids produced according to the methods of the present disclosure and a sample comprising a target nucleic acid. The combining is under conditions in which a target capture nucleic acid of the target capture nucleic acids specifically hybridizes to the target nucleic acid to produce a target capture nucleic acid-target nucleic acid complex. Such methods further comprise isolating the target capture nucleic acid-target nucleic acid complex.

The “conditions” during the combining step are those conditions in which a target capture nucleic acid specifically hybridizes to the target nucleic acid. Whether specific hybridization occurs is determined by such factors as the degree of complementarity between the relevant portion of the target capture nucleic acid (the reverse complement of the target nucleotide sequence) and the target nucleic acid, the length thereof, and the temperature at which the hybridization occurs, which may be informed by the melting temperatures (TM) of the relevant portion of the target capture nucleic acid and the target nucleic acid. The melting temperature refers to the temperature at which half of the target capture nucleic acids remain hybridized and half of the target capture nucleic acids dissociate into single strands. The Tm of a duplex may be experimentally determined or predicted using the following formula Tm=81.5+16.6(log 10[Na⁺])+0.41 (fraction G+C)−(600/N), where N is the chain length and [Na⁺] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of target capture nucleic acid/target nucleic acid duplexes depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

The target capture nucleic acids may be combined with any sample of interest comprising the target nucleic acid. In certain embodiments, the target nucleic acid is present in a nucleic acid sample isolated from a single cell, a plurality of cells (e.g., cultured cells), a tissue, an organ, or an organism (e.g., bacteria, yeast, or the like). According to some embodiments, the nucleic acid sample is isolated from a cell(s), tissue, organ, and/or the like of an animal. In some embodiments, the animal is a mammal, e.g., a mammal from the genus Homo (e.g., a human), a rodent (e.g., a mouse or rat), a dog, a cat, a horse, a cow, or any other mammal of interest. In certain embodiments, the nucleic acid sample is isolated/obtained from a source other than a mammal, such as bacteria, yeast, insects (e.g., drosophila), amphibians (e.g., frogs (e.g., Xenopus)), viruses, plants, or any other non-mammalian nucleic acid sample source.

According to some embodiments, the sample is a genomic DNA sample. In certain embodiments, the sample is an RNA sample, e.g., a total RNA sample, an mRNA sample, or the like. According to some embodiments, the sample is a complementary DNA (cDNA) sample. In certain embodiments, the sample is an ancient genomic DNA sample, a forensic nucleic acid sample, a circulating tumor DNA (ctDNA) sample (e.g., comprising ctDNAs isolated from a liquid biopsy), a cell-free DNA (cfDNA) sample (e.g., comprising cfDNAs isolated from blood or a fraction thereof), or an environmental DNA (eDNA) sample.

The nucleic acid sample may be from an extant organism or animal. In other embodiments, however, the nucleic acid sample may be from an extinct (or “ancient”) organism or animal, e.g., an extinct mammal, such as an extinct mammal from the genus Homo. According to some embodiments, the nucleic acid sample is obtained as part of a forensics analysis (e.g., a nucleic acid sample obtained from a crime scene, a victim of a crime, a crime suspect, and/or the like). In certain embodiments, the nucleic acid sample is obtained as part of a diagnostic analysis, e.g., from biopsy fluid or tissue (e.g., tumor biopsy tissue).

In certain embodiments, the nucleic acid sample comprises degraded DNA. Degraded DNA may be referred to as low-quality DNA or highly degraded DNA. Degraded DNA may be highly fragmented, and may include damage such as base analogs and abasic sites subject to miscoding lesions. For example, sequencing errors resulting from deamination of cytosine residues may be present in certain sequences obtained from degraded DNA, e.g., miscoding of C to T and G to A.

According to some embodiments, the nucleic acid sample is a cell-free nucleic acid sample, e.g., cell-free DNA, cell-free RNA, or both. Such cell-free nucleic acids may be obtained from any suitable source. In certain embodiments, the cell-free nucleic acids are from a body fluid sample selected from the group consisting of: whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. In certain embodiments, the cell-free nucleic acids are cell-free fetal DNAs. According to some embodiments, the cell-free nucleic acids are circulating tumor DNAs. In certain embodiments, the cell-free nucleic acids comprise infectious agent DNAs. According to some embodiments, the cell-free nucleic acids comprise DNAs from a transplant.

The term “cell-free nucleic acid” as used herein can refer to nucleic acid isolated from a source having substantially no cells. Cell-free nucleic acid may be referred to as “extracellular” nucleic acid, “circulating cell-free” nucleic acid (e.g., CCF fragments, ccf DNA) and/or “cell-free circulating” nucleic acid. Cell-free nucleic acid can be present in and obtained from blood (e.g., from the blood of an animal, from the blood of a human subject). Cell-free nucleic acid often includes no detectable cells and may contain cellular elements or cellular remnants. Non-limiting examples of acellular sources for cell-free nucleic acid are described above. Obtaining cell-free nucleic acid may include obtaining a sample directly (e.g., collecting a sample, e.g., a test sample) or obtaining a sample from another who has collected a sample. Without being limited by theory, cell-free nucleic acid may be a product of cell apoptosis and cell breakdown, which provides basis for cell-free nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded.

Cell-free nucleic acid can include different nucleic acid species, and therefore is referred to herein as “heterogeneous” in certain embodiments. For example, a sample from a subject having cancer can include nucleic acid from cancer cells (e.g., tumor, neoplasia) and nucleic acid from non-cancer cells. In another example, a sample from a pregnant female can include maternal nucleic acid and fetal nucleic acid. In another example, a sample from a subject having an infection or infectious disease can include host nucleic acid and nucleic acid from the infectious agent (e.g., bacteria, fungus, protozoa). In another example, a sample from a subject having received a transplant can include host nucleic acid and nucleic acid from the donor organ or tissue. In some instances, cancer, fetal, infectious agent, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, fetal, infectious agent, or transplant nucleic acid). In another example, heterogeneous cell-free nucleic acid may include nucleic acid from two or more subjects (e.g., a sample from a crime scene).

The nucleic acid sample may be a tumor nucleic acid sample (that is, a nucleic acid sample isolated from a tumor). “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like.

According to some embodiments, the nucleic acid sample is an environmental nucleic acid sample. In certain aspects, the environmental nucleic acid sample is a gaseous environmental nucleic acid sample. The gaseous environment may be, e.g., a stack gas, atmospheric air, indoor air, workplace atmosphere, landfill gas, industrial gas, exhaled breath, biogenic emissions, leaks from industrial installations, or the like. In certain embodiments, the environmental nucleic acid sample is a liquid environmental nucleic acid sample. The liquid environmental sample may be, e.g., drinking (or potable) water, surface water (e.g., river water, stream water, lake water, reservoir water, wetland water, bog water, or the like), ground water, waste water, well water, water from an unsaturated zone, rain water, run-off water, sea water, liquid industrial waste, sewage, surface films, or the like. In certain embodiments, the environmental nucleic acid sample is a solid environmental nucleic acid sample. The solid environmental sample may be from, e.g., ice, snow, soil, sewage sludge, bottom sediments, dust from electrofilters, vacuuming dust, plant material, forest floor, industrial waste, municipal waste, ashes, or the like.

In certain embodiments, the nucleic acid sample is pathogen DNA and/or RNA. Pathogens of interest include, but are not limited to, viral pathogens, bacterial pathogens, amoebic pathogens, parasitic pathogens, and fungal pathogens. According to some embodiments, the DNA is isolated from an infected host comprising the pathogen DNA and/or RNA. Infected hosts of interest include, but are not limited to, a terrestrial animal, a human, a terrestrial plant, an aquatic animal, and an aquatic plant. By “terrestrial” is meant an animal or plant that lives primarily on land (e.g., at least 75% of the time) as opposed to living in water. By “aquatic” is meant an animal or plant that lives primarily in water (e.g., at least 75% of the time) as opposed to on land. According to some embodiments, the DNA and/or RNA is isolated from excreta (e.g., urine and/or feces) of the infected host. In certain embodiments, the DNA and/or RNA is isolated from material shed from the infected host, non-limiting examples of which include hair and/or skin. Methods involving pathogen DNA and/or RNA and infected hosts may further comprise distinguishing the pathogen DNA and/or RNA from the infected host's DNA and/or RNA. Such methods may further include, subsequent to the distinguishing, analyzing the pathogen DNA and/or RNA, e.g., by sequencing as described in detail elsewhere herein.

Approaches, reagents and kits for isolating, purifying and/or concentrating DNA and RNA from sources of interest are known in the art and commercially available. For example, kits for isolating DNA from a source of interest include the DNeasy®, RNeasy®, QIAamp®, QIAprep® and QIAquick® nucleic acid isolation/purification kits by Qiagen, Inc. (Germantown, Md); the DNAzol®, ChargeSwitch®, Purelink®, GeneCatcher® nucleic acid isolation/purification kits by Life Technologies, Inc. (Carlsbad, CA); the NucleoMag®, NucleoSpin®, and NucleoBond® nucleic acid isolation/purification kits by Clontech Laboratories, Inc. (Mountain View, CA). In certain embodiments, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

When an organism, plant, animal, etc. from which the nucleic acid sample is obtained is extinct (or “ancient”), suitable strategies for recovering such nucleic acids are known and include, e.g., those described in Green et al. (2010) Science 328(5979):710-722; Poinar et al. (2006) Science 311(5759):392-394; Stiller et al. (2006) Proc. Natl. Acad. Sci. 103(37):13578-13584; Miller et al. (2008) Nature 456(7220):387-90; Rasmussen et al. (2010) Nature 463(7282):757-762; and elsewhere.

The methods of capturing target nucleic acids of the present disclosure further comprise isolating the target capture nucleic acid-target nucleic acid complex. According to some embodiments, the isolating comprises immobilizing the target capture nucleic acid-target nucleic acid complex on a solid support. The term “solid support” means an insoluble material having a surface to which reagents or materials can be attached so that they can be readily separated from a solution. In certain embodiments, the isolating comprises immobilizing target capture nucleic acid-target nucleic acid complexes on particulate solid supports. By “particulate solid supports” is meant a collection of solid supports having an average greatest dimension of 1000 micrometers (μm) or less. In certain embodiments, the collection of solid supports has an average greatest dimension of 750 μm or less, 500 μm or less, 250 μm or less, 100 μm or less, 1 μm or less, 0.75 μm or less, 0.50 μm or less, 0.25 μm or less, or 0.1 μm or less. In certain embodiments, the particulate solid supports have an average greatest dimension of from about 0.50 μm to about 500 μm, e.g., from about 0.75 μm to about 250 μm, e.g., about 1 μm.

A variety of materials can be used as solid supports. Support materials include any material that can act as a support for attachment of the target capture nucleic acid-target nucleic acid complexes. Suitable materials include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone, rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamides, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, metals, metalloids, magnetic materials, and any combinations thereof.

Particulate solid supports may be any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape.

In certain embodiments, the particulate solid supports are beads. As used herein, the term “bead” refers to a small mass that is generally spherical or spheroid in shape. According to some embodiments, a bead as used herein has an average diameter of from about 0.50 μm to about 500 μm, e.g., from about 0.75 μm to about 250 μm, e.g., about 1 μm.

Additionally, and for purposes herein, solid supports may be magnetically responsive, e.g., by virtue of comprising one or more paramagnetic and/or superparamagnetic substances, such as for example, magnetite. Such paramagnetic and/or superparamagnetic substances may be embedded within the matrix of a solid support, and/or may be disposed on an external and/or internal surface of a solid support.

In certain embodiments, particulate solid supports are particulate magnetic solid supports coated with a substance on their external surface that binds to binding member-labeled nucleotides of the target capture nucleic acids of the target capture nucleic acid-target nucleic acid complexes. According to some embodiments, the binding member-labeled nucleotides are biotin-labeled nucleotides and the substance comprises streptavidin or avidin.

A variety of suitable approaches may be employed to elute the target nucleic acids from the particulate solid supports, e.g., heat-denaturing the target capture nucleic acid-target nucleic acid complexes to dissociate the target nucleic acids from the target capture nucleic acids, exposing the complexes to a high salt solution to dissociate the target nucleic acids from the target capture nucleic acids, and/or the like.

According to some embodiments, the methods of the present disclosure of capturing target nucleic acids further comprise analyzing the captured and isolated target nucleic acids. The isolated target nucleic acids may be analyzed by a wide variety of types of analyses, including but not limited to, Southern analysis, Northern analysis, PCR analysis, and/or the like.

In certain embodiments, the methods of the present disclosure of capturing target nucleic acids further comprise sequencing all or a portion of a captured and isolated target nucleic acid. Sequencing platforms that may be employed to sequence such nucleic acids are available and include a sequencing platform provided by Illumina® (e.g., the HiSeq™, NextSeq™, MiSeq™ and/or NovaSeq™ sequencing systems); Oxford Nanopore™ Technologies (e.g., a SmidgION, MinION, GridION, or PromethION nanopore-based sequencing system), Ion Torrent™ (e.g., the Ion PGM™ and/or Ion Proton™ sequencing systems); Pacific Biosciences (e.g., a Sequel II ZMW-based sequencing system); Life Technologies™ (e.g., a SOLiD sequencing system); Roche (e.g., the 454 GS FLX+ and/or GS Junior sequencing systems); or any other sequencing platform of interest. Detailed design considerations and protocols for preparing nucleic acids (e.g., any necessary adapter addition, etc.), conducting nucleic acid sequencing runs, and analyzing the resulting sequencing data are provided by the manufacturers of such systems.

In nanopore sequencing, the nanopore serves as a biosensor and provides the sole passage through which an ionic solution on the cis side of the membrane contacts the ionic solution on the trans side. A constant voltage bias (trans side positive) produces an ionic current through the nanopore and drives ssDNA or ssRNA in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase. Because the ionic conductivity through the nanopore is sensitive to the presence of the nucleobase's mass and its associated electrical field, the ionic current levels through the nanopore reveal the sequence of nucleobases in the translocating strand. A patch clamp, a voltage clamp, or the like, may be employed.

Details for obtaining raw sequencing reads of nucleic acid molecules using nanopores are described, e.g., in Feng et al. (2015) Genomics, Proteomics & Bioinformatics 13(1):4-16. Nanopore-based sequencing systems are available and include the SmidgION, MinION, GridION, and PromethION nanopore-based sequencing systems available from Oxford Nanopore Technologies Limited. Detailed design considerations and protocols for performing nucleic acid sequencing are provided with such systems.

In zero mode waveguide (ZMW)-based sequence analysis, the ZMW is a nanoscale-sized well that serves as an optical confinement that allows observation of individual polymerase molecules. As a result, nucleotide incorporation events provide observation of an incorporating nucleotide analog that is readily distinguishable from non-incorporated nucleotide analogs. For a description of ZMWs and their application in nucleic acid sequencing, see, e.g., U.S. Patent Application Publication No. 2003/0044781 and U.S. Pat. No. 6,917,726, each of which is incorporated herein by reference in its entirety for all purposes. See also Levene et al. (2003) “Zero-mode waveguides for single-molecule analysis at high concentrations” Science 299:682-686, Eid et al. (2009) “Real-time DNA sequencing from single polymerase molecules” Science 323:133-138, and U.S. Pat. Nos. 7,056,676, 7,056,661, 7,052,847, 7,033,764, and 7,907,800, the full disclosures of which are incorporated herein by reference in their entirety for all purposes.

In the Illumina platform, the sequencing process involves clonal amplification of adaptor-ligated DNA fragments on the surface of a glass slide. Bases are read using a cyclic reversible termination strategy, which sequences the template strand one nucleotide at a time through progressive rounds of base incorporation, washing, imaging, and cleavage. In this strategy, fluorescently labeled 3′-O-azidomethyl-dNTPs are used to pause the polymerization reaction, enabling removal of unincorporated bases and fluorescent imaging to determine the added nucleotide. Following scanning of the flow cell with a coupled-charge device (CCD) camera, the fluorescent moiety and the 3′ block are removed, and the process is repeated.

Non-limiting examples of particular applications for which the methods of the present disclosure find use will now be described.

Targeted Sequencing of Cancer Genes

Cancer is a multigenic disease that arises due to mutations in multiple genes leading to dysregulation of cellular pathways. Ultra-deep sequencing is necessary to identify and validate mutations in cancer samples due to higher mutation rate and heterogeneity of tumor cell types. Mutational profile of cancer genes has been used in clinical diagnostics for personalized medicine. Multiple commercial kits are available for cancer target enrichment that target a few hundred genes either specific to an individual or common in all cancer types. Current cancer gene target enrichment reagents are expensive. The current average cost of target enrichment reagents for a 150 gene panel is ˜$100-$320 which limits their availability for a wide range of patients. The methods of the present disclosure enable the production of such reagents for a fraction of the current cost.

The Cancer Genome Atlas (TCGA) project has identified 299 most frequently mutated genes in all type of cancers. Memorial Sloan Kettering Cancer Center developed enrichment reagents for 341 cancer-relevant genes. The University of California, San Francisco (UCSF) has been sequencing about 500 cancer genes for diagnosis and personalized treatment options. In certain embodiments, target capture nucleic acids may be produced according to the methods of the present disclosure for these genes as well as other genes relevant to cancer immunotherapy and genes that predict the outcome of personalized medicine. Target capture nucleic acids may cover all canonical and non-canonical exons, exon-intron junctions as well as introns and regulatory regions that harbor actionable mutations and variations. Target capture nucleic acids can also be used for targeted RNAseq analysis. Target capture nucleic acids may include target regions for exon-exon junctions, isoform-specific exons, chimeric exons from gene fusion events, and alternative 3′ UTR regions in genes for which the expression is correlated with personalized treatment. Known gene-fusion targets may also be included.

SNP Enrichment Target Capture Nucleic Acids for Forensics Applications

A small set of polymorphic short tandem repeats (STRs) and SNPs have been widely used in forensic analysis, paternity tests, and victim identification. The Combined DNA Index System (CODIS) set of STRs is a well-known marker panel that has been used for decades. However, discriminatory power and paternity probability of STR based identification is poor in single-parent child identifications, identification of distant relatives, and identification without prior DNA information. Further, STRs have a high mutation rate that can obscure match identification. Hence, SNPs have been proposed either as alternative or augmented with STR analysis and high density SNPs are demonstrated for identification of individuals. SNPs are powerful for kinship analysis as they have very low mutation rates. SNPs informative for identifying individuals, ancestry, lineage and phenotypes have been identified and adopted for forensic analysis. Target enrichment and sequencing analysis of a few hundred SNPs have been developed for forensic applications. Though the forensic SNP panels and STR based CODIS search are useful in suspect identification, their application is limited in elusive cases, victim identification, kinship analysis, victim and missing person identification.

The recent boom in direct-to-consumer (DTC) genetic testing has resulted in the creation of large public databases of genetic information as well as family tree information. GEDmatch is a voluntary participation database that stores and shares DTC testing results for genealogy tracking purposes. Though the DTC testing panels were initially designed to track genealogy, a number of SNPs have been added to assess common traits and phenotypes. Current DTC services offer genotyping of about 600 to 700 thousand SNPs. Among the many DTC service providers, Ancerstry.com and 23andMe tested about 15 and 10 million people. Due to the higher number of SNPs being tested and the number of people whose genetic information is available in public and private databases, DTC test results have proven to be valuable in identifying suspects and victims of decade long cold cases. GEDmatch searching of crime site DNA helped to narrow suspects and solved four decade old crimes. According to some embodiments, a panel of target capture nucleic acids (e.g., about one million target capture nucleic acids) for biallelic SNPs is produced and/or employed for forensic testing using the methods of the present disclosure. The SNPs that are being tested in DTC panels may be combined with SNPs and STRs that have been used in forensic applications. Such high density genotyping will enable the identification of people who are distantly related as well as improve the discriminatory power and parental probability for kinship analysis and identification of missing person and victims.

### Compositions

Aspects of the present disclosure further include compositions. A composition of the present disclosure may include any of the reagents (e.g., nucleic acids, primers, enzymes, nucleotides, etc.) described elsewhere herein, in any desired combination. In certain embodiments, provided are compositions that comprise target capture nucleic acids produced according to any of the methods of the present disclosure.

Any of the compositions of the present disclosure may be present in a container. Suitable containers include, but are not limited to, tubes, vials, and plates (e.g., a 96- or other-well plate).

According to some embodiments, a composition of the present disclosure comprises target capture nucleic acids produced according to any of the methods of the present disclosure, and/or any desired combination of reagents (e.g., nucleic acids, primers, enzymes, nucleotides, etc.) present in a liquid medium. The liquid medium may be an aqueous liquid medium, such as water, a buffered solution, and the like. One or more additives such as a salt (e.g., NaCl, MgCl2, KCl, MgSO4), a buffering agent (a Tris buffer, N-(2-Hydroxyethyl)-piperazine-N′-(2-ethanesulfonic acid) (HEPES), 2-(N-Morpholino)-ethanesulfonic acid (MES), 2-(N-Morpholino)-ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

In some embodiments, a composition of the present disclosure is a lyophilized composition. A lyoprotectant may be included in such compositions in order to protect nucleic acids against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM. In certain aspects, a composition of the present disclosure is in a liquid form reconstituted from a lyophilized form. An example procedure for reconstituting a lyophilized composition is to add back a volume of pure water (typically equivalent to the volume removed during lyophilization); however solutions comprising buffering agents, antibacterial agents, and/or the like, may be used for reconstitution.

### Kits

Aspects of the present disclosure further include kits. In certain embodiments, a kit of the present disclosure includes any reagents (e.g., nucleic acids, primers, enzymes, nucleotides, etc.) described elsewhere herein, in any desired combination, and instructions for using the reagents to produce target capture nucleic acids in accordance with the methods of producing target capture nucleic acids of the present disclosure. According to some embodiments, such kits comprise a bridge oligonucleotide, one or more splint oligonucleotides, a rolling circle amplification primer, and a deoxynucleotide triphosphate (dNTP) mixture comprising modified nucleotides. According to some embodiments, a kit of the present disclosure comprises target capture nucleic acids produced according to any of the methods of the present disclosure, and instructions for using the target capture nucleic acids to capture target nucleic acids. Such kits may further include reagents and/or instructions for downstream analysis (e.g., sequencing) of the captured target nucleic acids.

Components of the kits may be present in separate containers, or multiple components may be present in a single container. A suitable container includes a single tube (e.g., vial), one or more wells of a plate (e.g., a 96-well plate, a 384-well plate, etc.), or the like.

Instructions included in a kit of the present disclosure may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:
1. A method of producing target capture nucleic acids, comprising:
   bidirectionally amplifying a circular nucleic acid template by rolling circle amplification (RCA) using first and second primers, wherein the circular nucleic acid template comprises a target nucleotide sequence and a restriction site, and wherein the bidirectional amplification produces a double-stranded concatemer comprising:
      a first strand comprising a plurality of linked units, each unit comprising the target nucleotide sequence and the restriction site; and
      a second strand which is the reverse complement of the first strand; and
   digesting the double-stranded concatemer using a restriction endonuclease that cleaves the restriction site to produce a plurality of restriction fragments, each restriction fragment comprising a target capture nucleic acid comprising the reverse complement of the target nucleotide sequence.
2. The method according to embodiment 1, wherein the first primer comprises a sequence that hybridizes to the restriction site.
3. The method according to embodiment 1 or embodiment 2, wherein the second primer comprises a sequence that hybridizes to the restriction site.
4. The method according to any one of embodiments 1 to 3, further comprising, prior to bidirectionally amplifying the circular nucleic acid template, producing the circular nucleic acid template by circularizing a linear nucleic acid comprising the target nucleotide sequence and the restriction site.
5. The method according to embodiment 4, wherein the circularizing is by splint ligation.
6. The method according to embodiment 5, comprising stabilizing the linear nucleic acid for splint ligation using a single-strand stabilizing protein.
7. The method according to embodiment 6, wherein the single-strand stabilizing protein is single-stranded nucleic acid binding protein (SSB).
8. The method according to any one of embodiments 5 to 7, wherein the linear nucleic acid comprises a poly dT domain at each of its ends, wherein the splint ligation comprises hybridizing a poly dA splint oligonucleotide to the poly dT domains, and wherein the circular nucleic acid template comprises a poly dA/poly dT site resulting from the splint ligation.
9. The method according to embodiment 8, wherein the first primer comprises a sequence that hybridizes to at least a portion of the poly dA/poly dT site.
10. The method according to embodiment 8 or embodiment 9, wherein the second primer comprises a sequence that hybridizes to at least a portion of the poly dA/poly dT site.
11. The method according to any one of embodiments 4 to 10, further comprising, prior to circularizing the linear nucleic acid, producing the linear nucleic acid.
12. The method according to embodiment 11, wherein producing the linear nucleic acid comprises attaching a nucleic acid comprising the restriction site to a nucleic acid comprising the target nucleotide sequence.
13. The method according to embodiment 12, wherein the attaching is by splint ligation.
14. The method according to embodiment 12 or embodiment 13, wherein the linear nucleic acid comprises a genomic DNA fragment.
15. The method according to embodiment 14, wherein the genomic DNA fragment is a bacterial artificial chromosome (BAC) DNA fragment.
16. The method according to embodiment 14 or embodiment 15, wherein producing the linear nucleic acid comprises:
   fragmenting genomic DNA to produce genomic DNA fragments;
   size-selecting the genomic DNA fragments, wherein the size-selected genomic DNA fragments comprise the genomic DNA fragment; and
   attaching a nucleic acid comprising the restriction site to the genomic DNA fragment.
17. The method according to any one of embodiments 1 to 16, wherein the double-stranded concatemer comprises 1000 or more of the linked units.
18. The method according to any one of embodiments 1 to 16, wherein the double-stranded concatemer comprises 100,000 or more of the linked units.
19. The method according to any one of embodiments 1 to 16, wherein the double-stranded concatemer comprises 1,000,000 or more of the linked units.
20. The method according to any one of embodiments 1 to 19, wherein the plurality of target capture nucleic acids comprise modified nucleotides incorporated into the double-stranded concatemer during the bidirectional amplification.
21. The method according to embodiment 20, wherein the modified nucleotides comprise binding member-labeled nucleotides.
22. The method according to embodiment 21, wherein the binding member-labeled nucleotides comprise biotin-labeled nucleotides.
23. The method according to any one of embodiments 20 to 22, wherein the modified nucleotides comprise thermostability-increasing nucleotides.
24. The method according to any one of embodiments 1 to 23, wherein the target nucleotide sequence is a target genomic DNA sequence, a target cell-free DNA (cfDNA) sequence, a target circulating tumor DNA (ctDNA) sequence, a target ribonucleic acid (RNA) sequence, or a target complementary DNA (cDNA) sequence.
25. Target capture nucleic acids produced according to the methods of any one of embodiments 1 to 24.
26. A method of capturing a target nucleic acid, comprising:
   combining the target capture nucleic acids of embodiment 25 and a sample comprising the target nucleic acid under conditions in which a target capture nucleic acid of the target capture nucleic acids specifically hybridizes to the target nucleic acid to produce a target capture nucleic acid-target nucleic acid complex; and
   isolating the target capture nucleic acid-target nucleic acid complex.
27. The method according to embodiment 26, wherein the sample is a genomic DNA sample.
28. The method according to embodiment 27, wherein the sample is an ancient genomic DNA sample.
29. The method according to embodiment 26, wherein the sample is a forensic nucleic acid sample.
30. The method according to embodiment 26, wherein the sample is a circulating tumor DNA (ctDNA) sample.
31. The method according to embodiment 30, wherein the ctDNA sample comprises ctDNAs isolated from a liquid biopsy.
32. The method according to embodiment 26, wherein the sample is a cell-free DNA (cfDNA) sample.
33. The method according to embodiment 32, wherein the cfDNA sample comprises cfDNAs isolated from blood or a fraction thereof.
34. The method according to embodiment 26, wherein the sample is an environmental DNA (eDNA) sample.
35. The method according to embodiment 26, wherein the sample is pathogen DNA.
36. The method according to embodiment 35, wherein the pathogen DNA is selected from the group consisting of: bacterial DNA, viral DNA, and parasite DNA.
37. The method according to the embodiment 35 or embodiment 36, wherein the DNA is isolated from an infected host comprising the pathogen DNA.
38. The method according to embodiment 37, wherein the infected host is selected from the group consisting of: a terrestrial animal, a human, a terrestrial plant, an aquatic animal, and an aquatic plant.
39. The method according to embodiment 37 or 38, wherein the DNA is isolated from a solid tissue sample, a body fluid sample, or excreta of the infected host.
40. The method according to embodiment 39, wherein the body fluid sample comprises blood, lymph, hemolymph, or a combination thereof.
41. The method according to embodiment 39, wherein the excreta comprises urine, feces, or a combination thereof.
42. The method according to embodiment 37 or 38, wherein the DNA is isolated from material shed from the infected host.
43. The method according to embodiment 42, material shed from the infected host is hair, fur, skin, exoskeleton, or a combination thereof.
44. The method according to any one of embodiments 37 to 43, further comprising distinguishing the pathogen DNA from the infected host's DNA.
45. The method according to embodiment 26, wherein the sample is an RNA sample.
46. The method according to embodiment 26, wherein the sample is a cDNA sample.
47. The method according to any one of embodiments 26 to 46, further comprising analyzing the target nucleic acid.
48. The method according to embodiment 47, wherein analyzing the target nucleic acid comprises sequencing all or a portion of the target nucleic acid.
49. A kit comprising:
   a bridge oligonucleotide;
   one or more splint oligonucleotides;
   a rolling circle amplification primer;
   a deoxynucleotide triphosphate (dNTP) mixture comprising modified nucleotides; and
   instructions for using the components of the kit to produce target capture nucleic acids according to the method of any one of embodiments 1 to 24.
50. A kit comprising:
   the target capture nucleic acids of embodiment 25; and
   instructions for using the target capture nucleic acids to capture a target nucleic acid.

The following examples are offered by way of illustration and not by way of limitation.

### EXPERIMENTAL

### Example 1—Production of Target Capture DNAs for Hypervariable Region 1 (HV1) and Hypervariable Region 2 (HV2) of Human Mitochondrial DNA (mtDNA)

As proof of principle, in this example, target capture DNAs (probes) that target hypervariable region 1 (HV1) and hypervariable region 2 (HV2) of human mitochondrial DNA (mtDNA) were produced. Hyper variable regions have high sequence diversity among populations and have been used for haplotyping the mitochondrial lineage. 13 tiling oligonucleotides (oligos) each 60 nucleotides (nt) long with 30 nt overlap with adjacent oligos were designed to cover HV1 on the University of California—Santa Cruz (UCSC) mitochondrial reference genome position 16000-16420 (SEQ ID Nos: 1-13 in the Table 1). All oligos also contained linker regions and a HindIII restriction site as schematically illustrated in FIG. 1 and synthesized by Integrated DNA Technologies (IDT). To demonstrate that multiple target capture DNAs (sometimes referred to herein as “probes” or “baits”) can be generated from one long oligonucleotide, 6 bait regions each 48 bp long and gapped apart by 10 bp were designed to cover HV2 on UCSC mitochondrial reference genome position 50-388. Two 199 bp oligos were synthesized by concatenating 3 target regions per oligo (SEQ ID Nos: 14 and 15 in the Table 1). The target regions are flanked by AscI recognition site and 8-10 Ts. Both oligos also contained linker regions as schematically illustrated in FIG. 1 and synthesized by IDT.

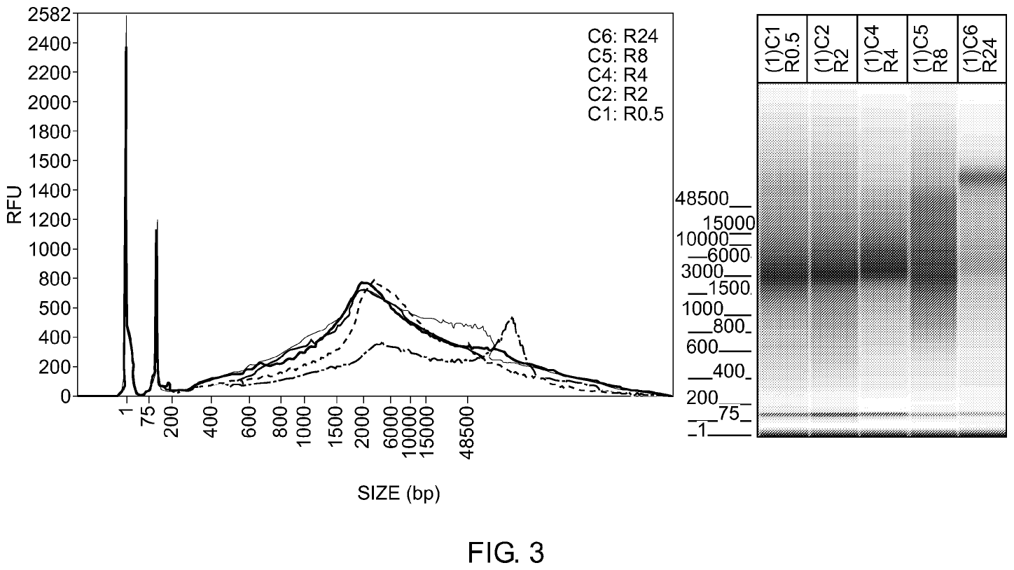

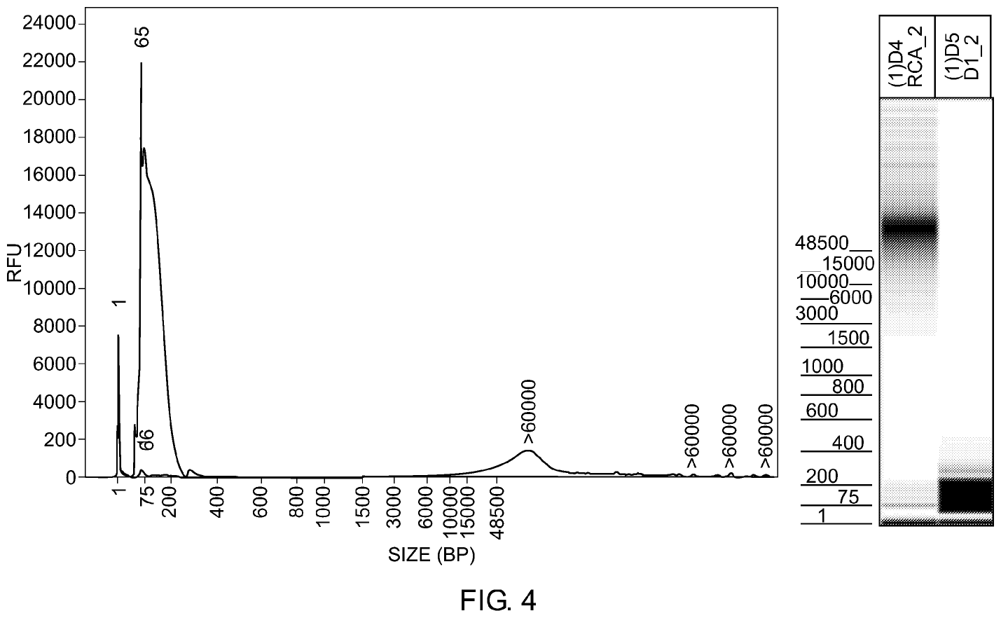

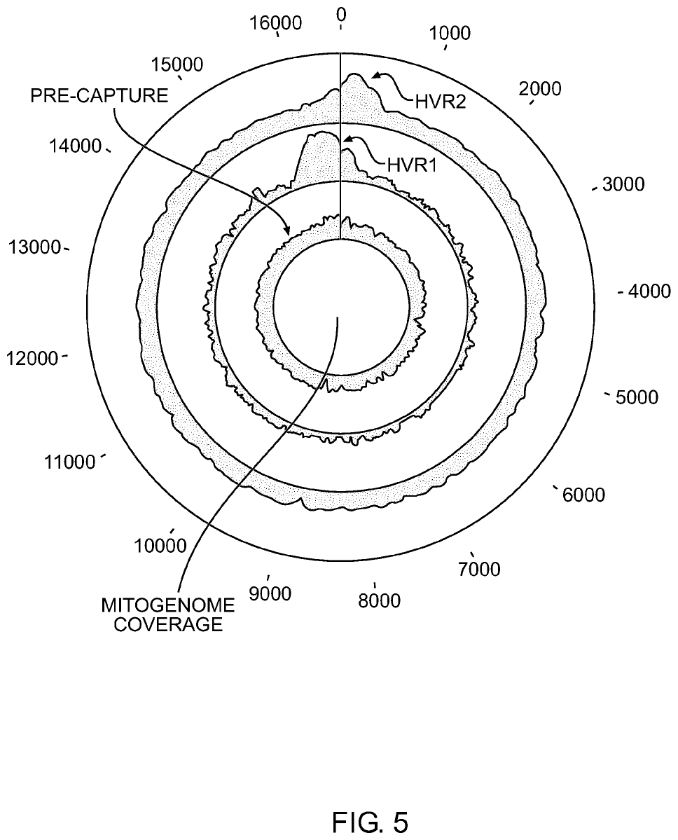

Target oligos are circularized by splint ligation using poly-dA oligos as splint (SEQ ID NO: 16). Circularized oligo templates isothermally amplified using Phi29 DNA polymerase with appropriate primers (SEQ ID NOs: 17-20) for 24 hours produced high molecular weight (HMW)-DNA as shown by capillary electrophoresis (FIG. 3). The time-dependent increase in average size and concentration of the RCA product indicated linear amplification. Restriction digestion of RCA products annealed with oligo primers produced near complete digestion that produced monomeric probes of ˜80 nt size (FIG. 4).

TABLE 1
Oligonucleotide Sequences

| | Sequence 5′ -> 3′ | Notes |
|---|---|---|
| SEQ ID NO: 1 | TTTTTTTGATTCTAATTTAAACTATTCTCTGTTCTTTCATGGGG | chrM:16000-16060 |
| | AAGCAGATTTGGGTACCACCCAAGGCGCGATCAAGCTTTTTTTT | |
| SEQ ID NO: 2 | TTTTTTTCATGGGGAAGCAGATTTGGGTACCACCCAAGTATTGA | chrM:16030-16090 |
| | CTCACCCATCAACAACCGCTATGTGCGCGATCAAGCTTTTTTTT | |
| SEQ ID NO: 3 | TTTTTTTGTATTGACTCACCCATCAACAACCGCTATGTATTTCG | chrM:16060-16120 |
| | TACATTACTGCCAGCCACCATGAAGCGCGATCAAGCTTTTTTTT | |
| SEQ ID NO: 4 | TTTTTTTTATTTCGTACATTACTGCCAGCCACCATGAATATTGT | chrM:16090-16150 |
| | ACGGTACCATAAATACTTGACCACGCGCGATCAAGCTTTTTTTT | |
| SEQ ID NO: 5 | TTTTTTTATATTGTACGGTACCATAAATACTTGACCACCTGTAG | chrM:16120-16180 |
| | TACATAAAAACCCAATCCACATCAGCGCGATCAAGCTTTTTTTT | |
| SEQ ID NO: 6 | TTTTTTTCCTGTAGTACATAAAAACCCAATCCACATCAAAACCC | chrM:16150-16210 |
| | CCTCCCCATGCTTACAAGCAAGTAGCGCGATCAAGCTTTTTTTT | |
| SEQ ID NO: 7 | TTTTTTTAAAACCCCCTCCCCATGCTTACAAGCAAGTACAGCAA | chrM:16180-16240 |
| | TCAACCCTCAACTATCACACATCAGCGCGATCAAGCTTTTTTTT | |
| SEQ ID NO: 8 | TTTTTTTACAGCAATCAACCCTCAACTATCACACATCAACTGCA | chrM:16210-16270 |
| | ACTCCAAAGCCACCCCTCACCCACGCGCGATCAAGCTTTTTTTT | |
| SEQ ID NO: 9 | TTTTTTTAACTGCAACTCCAAAGCCACCCCTCACCCACTAGGAT | chrM:16240-16300 |
| | ACCAACAAACCTACCCACCCTTAAGCGCGATCAAGCTTTTTTTT | |
| SEQ ID NO: 10 | TTTTTTTCTAGGATACCAACAAACCTACCCACCCTTAACAGTAC | chrM:16270-16330 |
| | ATAGTACATAAAGCCATTTACCGTGCGCGATCAAGCTTTTTTTT | |
| SEQ ID NO: 11 | TTTTTTTACAGTACATAGTACATAAAGCCATTTACCGTACATAG | chrM:16300-16360 |
| | CACATTACAGTCAAATCCCTTCTCGCGCGATCAAGCTTTTTTTT | |
| SEQ ID NO: 12 | TTTTTTTTACATAGCACATTACAGTCAAATCCCTTCTCGTCCCC | chrM:16330-16390 |
| | ATGGATGACCCCCCTCAGATAGGGGCGCGATCAAGCTTTTTTTT | |
| SEQ ID NO: 13 | TTTTTTTCGTCCCCATGGATGACCCCCCTCAGATAGGGGTCCCT | chrM:16360-16420 |
| | TGACCACCATCCTCCGTGAAATCAGCGCGATCAAGCTTTTTTTT | |
| SEQ ID NO: 14 | TTTTTTTGGTATTTTCGTCTGGGGGGTATGCACGCGATAGCATT | chrM: 50-98, |
| | GCGAGACGCGGCGCGCCTTTTTTTTTAGCACCCTATGTCGCAGT | 108-156, 166-214 |
| | ATCTGTCTTTGATTCCTGCCTCATCCTATTAGGCGCGCCTTTTT | |
| | TTTTCCTACGTTCAATATTACAGGCGAACATACTTACTAAAGTG | |
| | TGTTAATTAGGCGCGCCTTTTTT | |
| SEQ ID NO: 15 | TTTTTGTAGGACATAATAATAACAATTGAATGTCTGCACAGCCA | chrM: 224-272, |
| | CTTTCCACAGGCGCGCCTTTTTTTTTTAACAAAAAATTTCCACC | 282-330, 340-388 |
| | AAACCCCCCCTCCCCCGCTTCTGGCCACAGCGGCGCGCCTTTTT | |
| | TTTTCATCTCTGCCAAACCCCAAAAACAAAGAACCCTAACACCA | |
| | GCCTAACCAGGCGCGCCTTTTTT | |
| SEQ ID NO: 16 | /5AmMC6/AAAAAAAAAAAA/3AmMO/ | Splint oligonucleotide |
| SEQ ID NO: 17 | GCGCGATCAAGCTTTTTTTTT | RCA_Hind3_rev |
| | | primer |
| SEQ ID NO: 18 | AAAAAAAAAGCTTGATCGCGC | RCA_Hind3_fwd |
| | | primer |
| SEQ ID NO: 19 | GGCGCGCCTTTTTTTTT | RCA_Ascl_rev primer |
| SEQ ID NO: 20 | AAAAAAAAAGGCGCGCC | RCA_Ascl_fwd |
| | | primer |
| SEQ ID NO: 21 | GCGCGATCAAGCTTTTTTTTTTTTTTTTTTTTT | WGE_bridge_oligo_v1 |
| SEQ ID NO: 22 | /5AmMC6/GCTTGATCGCGCNNNNNNNN/3AmMO/ | WGE_Splint-1_v1 |
| SEQ ID NO: 23 | /5AmMC6/NNNNNNNNAAAAAAAAAAA/3AmMO/ | WGE_Splint-1_v1 |
| SEQ ID NO: 24 | GGCGCGCCTTTTTTTTTTTTTTTGC | WGE_bridge_oligo_v2 |
| SEQ ID NO: 25 | /5AmMC6/NNNNNNNNGCAAAAAAAAAAAAAAAGGCGCGCCNNN | WGE_Splint_v2 |
| | NNNNN/3AmMO/ | |

Baits were hybridized at 65° C. for 18 hours with next generation sequencing (NGS) libraries prepared using hair DNA. Pre- and post-capture libraries were sequenced and the read coverage depth for the mitochondrial genome are shown in the table below.

TABLE 2

| | | Whole | HV1 | HV1 | HV2 | HV2 |
|---|---|---|---|---|---|---|
| | Raw | Mitochondria | region | enrichment | region | enrichment |
| Conditions | reads | coverage | coverage | ratio | coverage | ratio |
| Pre-capture libraries | 1,639,429 | 52 | 157 | 2.99 | 101 | 1.92 |
| Captured with HV1 baits | 347,338 | 767 | 22,127 | 28.85 | 273 | 0.36 |
| Captured with HV2 baits | 726,425 | 1,011 | 151 | 0.15 | 43,530 | 43.07 |

The HV1 region was covered with an average coverage of 22,127×, whereas the coverage for the whole mitochondrial genome was 767×, indicating ˜29-fold enrichment for HV1 region. The same library captured with HV2 probes generated libraries enriched for HV2 region covered with an average coverage of 43,530× and 1,011× coverage for whole mitochondria, indicating ˜43 fold enrichment for the HV2 region. These experiments demonstrate that baits synthesized using the strategy are efficient for target enrichment and can be used for ultra-deep sequencing of one or more regions of interest.

### Example 2—Production and Validation of Target Capture Probes for Entire Human Mitochondrial DNA (mtDNA) and Forensically-Relevant SNPs for Forensic Analyses

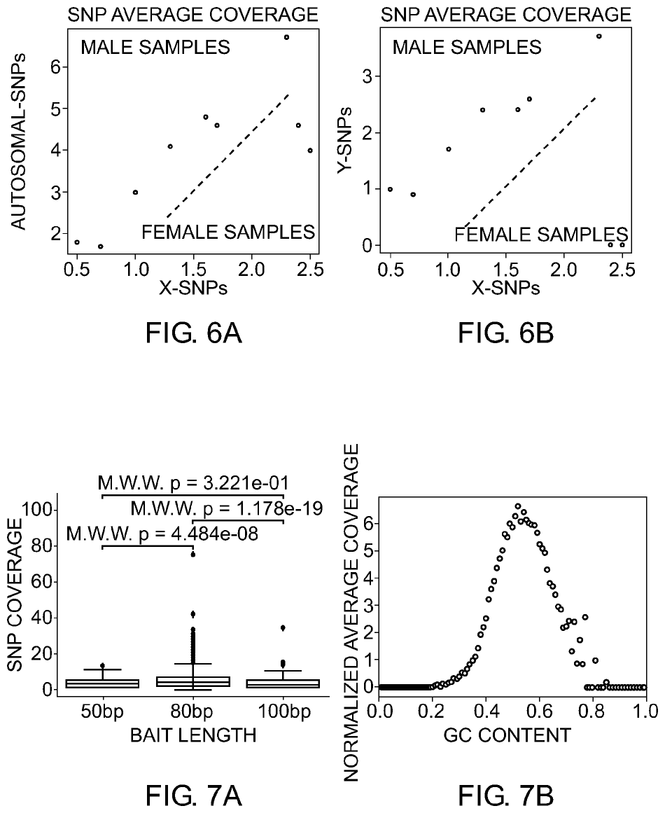

To demonstrate the feasibility of the probe generation method for human forensic application, ˜2000 forensically relevant SNPs and STRs from the ALFRED database (Rajeevan H et al, Nucleic Acids Res. 2012 January; 40: D1010-5) was designed as a panel. The panel consists of one probe for each of the 896 autosomal SNPs, 33 X chromosomal SNPs, 651 Y chromosomal SNPs, 170 probes targeting 170 micro haplotypes, 40 probes to cover 20 CODIS STR regions and 180 probes targeting the entire human mtDNA, in total 1970 probes. The panel was made from a pool of 1970 oligonucleotide probe templates. Oligo templates were circularized, isothermally amplified by RCA and digested with restriction enzymes to generate probes. DNA isolated from the saliva of 8 volunteers was made into an NGS library using the single strand adapter ligation method (Troll C J et al, BMC Genomics. 2019 Dec. 27; 20(1):1023.). Libraries were captured with 10 ng of probes by hybridization at 65° C. for 18 hours. Post-capture libraries were sequenced on Illumina NextSeq for ˜500 k raw reads per sample. On average, 86.6% unique reads remained after adapter trimming and merging of overlapping pairs, of which 45.2% mapped to mitochondria resulting in 2934× average coverage (864×-3255×). About 83% of the autosomal SNPs targets are covered on average at 3.9× reads (1.8-6.7×), and 16.8% (8.7%-31.1%) SNP targets had zero coverage. Similarly, ˜60% of X-SNPs are covered at 13× (9×-21×). About two-thirds of the targeted Y-SNPs are covered at 2.1× (0×-3.7×) and one female sample had no coverage across all Y-SNPs and the female NIST standard (NA12878) also had no Y-SNP coverage. Plotting SNP coverage for autosomes and X chromosome (FIG. 6, panel A) as well as between X and Y chromosome (FIG. 6, panel B) distinguished male and female samples. Overall, 78.3% of the targeted regions were covered by at least one read. Higher coverage of mtDNA versus SNPs was due to the proportion of nuclear to mtDNA in the input DNA. The forensic panel contains one probe per target and hence abundant mtDNA in input material with excess probe molecules in the capture reaction resulted in over-enrichment of mtDNA.

TABLE 3
Forensic SNPs and whole mitochondria capture results

| | | | Mitochondria | Autosomal | X-SNP | Y-SNP | % Zero |
|---|---|---|---|---|---|---|---|
| | Reads | Unique | average | SNP average | average | average | coverage |
| Samples | (after merge) | reads | coverage | coverage | coverage | coverage | targets |
| Male_Donor_1 | 195,945 | 89.0% | 864.5 | 1.8 | 0.5 | 1.0 | 35.8% |
| Male_Donor_2 | 209,055 | 87.3% | 1138.5 | 1.7 | 0.7 | 0.9 | 38.4% |
| Male_Donor_3 | 417,867 | 88.1% | 2073.7 | 3.0 | 1.0 | 1.7 | 23.4% |
| Male_Donor_4 | 508,964 | 87.2% | 2516.6 | 4.1 | 1.3 | 2.4 | 17.3% |
| Male_Donor_5 | 541,000 | 86.5% | 2382.3 | 6.7 | 2.3 | 3.7 | 12.0% |
| Male_Donor_6 | 582,364 | 84.7% | 3020.0 | 4.8 | 1.6 | 2.4 | 18.1% |
| Male_Donor_7 | 604,855 | 83.1% | 3255.4 | 4.6 | 1.7 | 2.6 | 16.1% |
| Female_Donor_1 | 585,717 | 86.9% | 3021.3 | 4.6 | 2.4 | 0.0 | 12.7% |
| Female_NA12878 | 1,458,110 | 80.1% | 8134.7 | 4.0 | 2.5 | 0.0 | 12.0% |

### Example 3—Production and Validation of Target Capture Probes for Targeted Genotyping of Ancient Horse DNA

To demonstrate the feasibility of the probe generation method for ancient DNA analysis, a horse SNP panel was designed. Wider interest in the evolution and population history of horses and related species motivated to design a SNP panel to genotype the Equidae family. 22,847 SNPs and chrY target regions in the Horse genome (EquCab2) were chosen based on their neutral evolution and Mendelian characteristics. The panel was designed with one 80 bp long probe per target centered at the SNP position. Probes of 50 bp, 80 bp and 100 bp lengths were also designed with non-overlapping SNP targets to test the effect of probe length on coverage. A final panel containing 23,999 Horse_SNP probe templates was synthesized as an oligo pool. The oligo pool was circularized, isothermally amplified and digested with restriction enzymes as described in the Methods section to generate probes. NGS libraries made from DNA isolated from mustang (Equus ferus) blood were captured with 50 ng Horse_SNP probes in four different hybridization buffers (HybBuf). HybBuf 1 contains 100 mM MES pH 6.5 and 5M NaCl, HybBuf 2 contains 6×SSC pH 7.0, HybBuf 3 contains 6×SSPE pH 7.5 and HybBuf 4 contains 100 mM Tris pH 8.0 and 5M NaCl. All buffers also contain 0.1% SDS, 10 mM EDTA and 10% DMSO at final concentration. Sequencing results show that HybBuf4 produces 44.9% percent selected bases, highest among all the buffers tested (Table 4). Regardless of the hybridization buffers, SNPs targeted with 80 bp long probes have 2-fold higher coverage than SNPs targeted by both 50 bp and 100 bp long probes (FIG. 7, panel A). In addition, probes with 40-70% GC content produce higher SNP coverage (FIG. 7, panel B). DNA isolated from more than 10,000 year-old ancient horse bone samples were made in libraries using a single strand library preparation method (Troll C J et al, BMC Genomics. 2019 Dec. 27; 20(1):1023.). 100-150 ng of the libraries were hybridized with 25-75 ng Horse_SNP probes at 50° C. for 48 hrs. Post-capture libraries were sequenced for ˜1M raw reads resulting in 2.9× average coverage (0.2×-5.5×) of 74% targets and 26% (5%-83%) of targeted SNPs were not covered.

TABLE 4
Horse SNP capture results

| | | | % | Mean | Median | Max | % Zero | | |
|---|---|---|---|---|---|---|---|---|---|
| | Endogenous | Total | Selected | Target | Target | Target | Coverage | % Target | % Target |
| Sample | content | Reads | Bases | Coverage | Coverage | Coverage | Targets | Bases at 1X | Bases at 10X |
| Mustang_HybBuf1_pH 6.5 | 100.0% | 589,343 | 40% | 4.3 | 3 | 41 | 21% | 79% | 13% |
| Mustang_HybBuf2_pH 7.0 | 100.0% | 532,522 | 39% | 3.4 | 2 | 73 | 25% | 75% | 7% |
| Mustang_HybBuf3_pH 7.4 | 100.0% | 571,850 | 41% | 3.6 | 2 | 71 | 26% | 74% | 9% |
| Mustang_HybBuf4_pH 8.0 | 100.0% | 495,338 | 45% | 3.6 | 2 | 70 | 24% | 76% | 9% |
| Ancient_horse_sample_1 | 3.8% | 981,982 | 15% | 0.2 | 0 | 5 | 83% | 17% | 0% |
| Ancient_horse_sample_2 | 11.3% | 1,233,736 | 21% | 2.6 | 2 | 46 | 17% | 83% | 1% |
| Ancient_horse_sample_3 | 23.1% | 696,836 | 19% | 1.7 | 1 | 22 | 24% | 76% | 0% |
| Ancient_horse_sample_4 | 42.0% | 797,375 | 23% | 2.7 | 2 | 36 | 18% | 82% | 1% |
| Ancient_horse_sample_5 | 74.7% | 925,277 | 17% | 4.1 | 4 | 38 | 10% | 90% | 6% |
| Ancient_horse_sample_6 | 77.6% | 1,138,136 | 20% | 5.8 | 5 | 70 | 5% | 95% | 15% |

### Example 4—Production and Validation of Whole Genome Enrichment Probes

DNA samples isolated from ancient specimens, forensic exhibits and environmental samples often contaminated with unwanted DNA. Therefore, it is important to enrich the genomes of interest to reduce the sequencing cost. Probes targeting the entire genome of an organism are used for whole genome enrichment (WGE). Human and horse probes are generated to demonstrate the WGE probe generation Methods exemplified in FIG. 2. To generate human WGE probes, 1 ug of genomic DNA isolated from the GM12878 cells were nicked with 0.02 U DNase I for 15 min at 15 C. Nicked DNA denatured at 95 C for 5 min to generate single stranded DNA. Oligos containing poly A tail and restriction enzyme recognition site (WGE_bridge_oligo_v1, SEQ ID NO: 21 in Table 1) were annealed with oligos containing randomers (WGE_Splint-1_v1 and WGE_Splint-2_v1, SEQ ID NOs: 22 and 23 in Table 1). 100 ng of ssDNA genomic fragments were ligated with 3 pmol of annealed oligos in a reaction containing 2000 U of T4 DNA ligase and 10 U T4 PNK enzymes with 15% PEG8000 at 37 C for 1 hr and then 25 C for 3 hr. Circularized genomic fragments were denatured to remove splint oligos and amplified by RCA. RCA reaction contained 30 U of phi29 polymerase, 25 nmol of dNTP mix, 2 nmol each of biotin-11-dATP and biotin-11-dUTP, 25 pmol each of RCA_Hind3_fwd and RCA_Hind3_rev (SEQ ID NO: 17 and 18) primers in 1×Phi29 buffer with BSA and DTT. RCA was performed at 30 C for 40 hr and the amplified products were digested with 100 U of HindIII restriction enzyme at 37 C for 3 hr. Digested RCA products were cleaned with 2.5×SPRI beads, yielding 4020 ng of probes which were aliquoted and stored in −20 C. In a separate experiment, horse WGE probes were made using Equine Horse Genomic DNA purchased from Zyagen, yielding 3840 ng of horse WGE probes.

To demonstrate WGE using the above generated probes, two NGS libraries containing unique Illumina dual indices were generated for human and horse DNA. In the first experiment, 180 ng of human library and 20 ng of horse library were mixed and captured with 100 ng of horse WGE probes. Without enrichment, the horse library was expected to represent only 10% of the total reads. However, sequencing of post-capture library produced 4,138,296 raw reads, of which 3,798,652 reads belonged to horse library as identified by the unique dual indices representing 91.8% of total reads, a 9.18-fold enrichment. Only 339,644 reads containing human library dual indices were identified, which was 8.2% of total reads. Alignment of the horse library to Equine reference genome (EquCab2) resulted in 106.6% total mapped reads. Higher percentage of mapped reads was due to secondary alignment in low-complexity regions. For the second experiment, 40 ng of human library and 160 ng of horse library were mixed with an expectation of 1:4 ratio and captured with 120 ng of human WGE probes. Sequencing of post-capture library resulted in 3,181,708 raw reads with human indices (95.7%) and 141,354 (4.3%) reads with horse indices, representing a 4.79-fold enrichment of human DNA. Human data was aligned with the hg38 reference genome resulting in 100.6% mapped reads. Results of the WGE enrichment experiments are summarized in Table 5.

TABLE 5
Summary of Whole Genome Enrichment Experiments

| | | Pre- | Post- | Post- | |
|---|---|---|---|---|---|
| | | en- | en- | enrichment | Fold |
| Experi- | Spe- | richment | richment | raw read | en- |
| ment | cies | ratio | raw reads | percent | richment |
| WGE of | Horse | 10% | 3,798,652 | 91.8% | 9.18x |
| horse DNA | Human | 90% | 339,644 | 8.2% | — |
| WGE of | Horse | 80% | 141,354 | 4.3% | — |
| human | Human | 20% | 3,181,708 | 95.7% | 4.79x |
| DNA | | | | | |

### Example 5— Production of Whole Genome Enrichment Probes for Intracellular Pathogens

DNA information of intracellular pathogens including bacteria, viruses and protozoan parasites are difficult to isolate from their host cells. Distinguishing host and parasite DNA and identification of DNA from intracellular pathogens is an important task for disease diagnostics and control. Current methods of intracellular pathogen identification involve PCR amplification of small regions in the pathogen genome. However, discriminating closely related species and identification of drug resistance can't be achieved by PCR amplification of ID regions, but by sequence analyzing the whole genome. Whole genome enrichment (WGE) probes can enrich intracellular pathogens' DNA from their host DNA. Toxoplasmosis is a human vector borne infection caused by Toxoplasma gondii, an intracellular parasite with felines as primary hosts. To demonstrate the WGE probe generation for T. gondii, DNA was isolated from lab cultured parasites and 1 ug of genomic DNA was sheared using 0.02 U DNAse I for 15 min at 15 C. Sheared DNA denatured at 95° C. for 5 min to generate single stranded DNA. Bridge oligo containing poly T tail and AscI restriction enzyme recognition site (WGE_bridge_oligo_v2, SEQ ID NO: 24) is annealed with oligos containing randomers (WGE_Splint_v2, SEQ ID NO: 25). In separate experiments, 100 ng of ssDNA genomic fragments are ligated with 35 pmol of annealed v1 or v2 WGE_bridge oligos using two different ligation buffer conditions, both containing 2000 U of T4 DNA ligase and 10 U T4 PNK enzymes 37 C for 1 hr and then 25 C for 3 hr. One reaction contained 20% PEG8000 and 26.24 ng/ul SSB at final concentration and another reaction without PEG and SSB. Circularized genomic fragments are denatured to remove splint oligos and amplified by RCA. The RCA reaction contained 30 U of phi29 polymerase, 25 nmol of dNTP mix, 2 nmol each of biotin-11-dATP and biotin-11-dUTP, 300 pmol appropriate RCA primers (SEQ ID NO: 17-20) in 1×Phi29 buffer with BSA and DTT. RCA was performed at 30° C. for 46 hr and the amplified products are digested with 1000 of either HindIII or AscI restriction enzyme at 37° C. for 6 hr. Digested RCA products were cleaned with 2×SPRI beads to make probes and final probe yields are summarized in Table 6. Toxoplasma probes can be used to detect T. gondii in human samples, DNA isolated from animals and environmental DNA samples.

TABLE 6
WGE probes yield using different circularization reactions.

| | | Probe | |
|---|---|---|---|
| Bridge oligos | Buffer condition | yield | |
| WGE_bridge_oligo_v1 | 1X T4 DNA ligase reaction | 3,000 | ng |
| with two splint oligos | without PEG or SSB | | |
| WGE_bridge_oligo_v1 | 1X T4 DNA ligase reaction with | 1,260 | ng |
| with two splint oligos | 20% PEG and 26.24 ng/ul SSB | | |
| WGE_bridge_oligo_v2 | 1X T4 DNA ligase reaction | 288 | ng |
| with one splint oligos | without PEG or SSB | | |
| WGE_bridge_oligo_v2 | 1X T4 DNA ligase reaction with | 378 | ng |

| | |
|---|---|
| with one splint oligos | 20% PEG and 26.24 ng/ul SSB |

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of producing target capture nucleic acids, comprising:
bidirectionally amplifying a circular nucleic acid template by rolling circle amplification (RCA) using first and second primers, wherein the circular nucleic acid template comprises a target nucleotide sequence and a restriction site, and wherein the bidirectional amplification produces a double-stranded concatemer comprising:
a first strand comprising a plurality of linked units, each unit comprising the target nucleotide sequence and the restriction site; and
a second strand which is the reverse complement of the first strand; and
digesting the double-stranded concatemer using a restriction endonuclease that cleaves the restriction site to produce a plurality of restriction fragments, each restriction fragment comprising a target capture nucleic acid comprising the reverse complement of the target nucleotide sequence.

2. The method according to claim 1, wherein the first primer comprises a sequence that hybridizes to the restriction site.

3. The method according to claim 1, wherein the second primer comprises a sequence that hybridizes to the restriction site.

4. The method according to claim 1, further comprising, prior to bidirectionally amplifying the circular nucleic acid template, producing the circular nucleic acid template by circularizing a linear nucleic acid comprising the target nucleotide sequence and the restriction site.

5. The method according to claim 4, wherein the circularizing is by splint ligation.

6. The method according to claim 5, comprising stabilizing the linear nucleic acid for splint ligation using a single-strand stabilizing protein.

7. The method according to claim 6, wherein the single-strand stabilizing protein is single-stranded nucleic acid binding protein (SSB).

8. The method according to claim 5, wherein the linear nucleic acid comprises a poly dT domain at each of its ends, wherein the splint ligation comprises hybridizing a poly dA splint oligonucleotide to the poly dT domains, and wherein the circular nucleic acid template comprises a poly dA/poly dT site resulting from the splint ligation.

9. The method according to claim 8, wherein the first primer comprises a sequence that hybridizes to at least a portion of the poly dA/poly dT site.

10. The method according to claim 8, wherein the second primer comprises a sequence that hybridizes to at least a portion of the poly dA/poly dT site.

11. The method according to claim 4, further comprising, prior to circularizing the linear nucleic acid, producing the linear nucleic acid.

12. The method according to claim 11, wherein producing the linear nucleic acid comprises attaching a nucleic acid comprising the restriction site to a nucleic acid comprising the target nucleotide sequence.

13. The method according to claim 12, wherein the attaching is by splint ligation.

14. The method according to claim 12, wherein the linear nucleic acid comprises a genomic DNA fragment.

15. The method according to claim 14, wherein the genomic DNA fragment is a bacterial artificial chromosome (BAC) DNA fragment.

16. The method according to claim 14, wherein producing the linear nucleic acid comprises:
fragmenting genomic DNA to produce genomic DNA fragments;
size-selecting the genomic DNA fragments, wherein the size-selected genomic DNA fragments comprise the genomic DNA fragment; and
attaching a nucleic acid comprising the restriction site to the genomic DNA fragment.

17. The method according to claim 1, wherein the double-stranded concatemer comprises 1000 or more of the linked units.

18. The method according to claim 1, wherein the double-stranded concatemer comprises 100,000 or more of the linked units.

19. The method according to claim 1, wherein the double-stranded concatemer comprises 1,000,000 or more of the linked units.

20. The method according to claim 1, wherein the plurality of target capture nucleic acids comprise modified nucleotides incorporated into the double-stranded concatemer during the bidirectional amplification.

21. The method according to claim 20, wherein the modified nucleotides comprise binding member-labeled nucleotides.

22. The method according to claim 21, wherein the binding member-labeled nucleotides comprise biotin-labeled nucleotides.

23. The method according to claim 20, wherein the modified nucleotides comprise thermostability-increasing nucleotides.

24. The method according to claim 1, wherein the target nucleotide sequence is a target genomic DNA sequence, a target cell-free DNA (cfDNA) sequence, a target circulating tumor DNA (ctDNA) sequence, a target ribonucleic acid (RNA) sequence, or a target complementary DNA (cDNA) sequence.

25. Target capture nucleic acids produced according to the methods of claim 1.

26. A method of capturing a target nucleic acid, comprising:
combining the target capture nucleic acids of claim 25 and a sample comprising the target nucleic acid under conditions in which a target capture nucleic acid of the target capture nucleic acids specifically hybridizes to the target nucleic acid to produce a target capture nucleic acid-target nucleic acid complex; and
isolating the target capture nucleic acid-target nucleic acid complex.

27. The method according to claim 26, wherein the sample is a genomic DNA sample.

28. The method according to claim 27, wherein the sample is an ancient genomic DNA sample.

29. The method according to claim 26, wherein the sample is a forensic nucleic acid sample.

30. The method according to claim 26, wherein the sample is a circulating tumor DNA (ctDNA) sample.

31. The method according to claim 30, wherein the ctDNA sample comprises ctDNAs isolated from a liquid biopsy.

32. The method according to claim 26, wherein the sample is a cell-free DNA (cfDNA) sample.

33. The method according to claim 32, wherein the cfDNA sample comprises cfDNAs isolated from blood or a fraction thereof.

34. The method according to claim 26, wherein the sample is an environmental DNA (eDNA) sample.

35. The method according to claim 26, wherein the sample is pathogen DNA.

36. The method according to claim 35, wherein the pathogen DNA is selected from the group consisting of: bacterial DNA, viral DNA, and parasite DNA.

37. The method according to the claim 35, wherein the DNA is isolated from an infected host comprising the pathogen DNA.

38. The method according to claim 37, wherein the infected host is selected from the group consisting of: a terrestrial animal, a human, a terrestrial plant, an aquatic animal, and an aquatic plant.

39. The method according to claim 37, wherein the DNA is isolated from a solid tissue sample, a body fluid sample, or excreta of the infected host.

40. The method according to claim 39, wherein the body fluid sample comprises blood, lymph, hemolymph, or a combination thereof.

41. The method according to claim 39, wherein the excreta comprises urine, feces, or a combination thereof.

42. The method according to claim 37, wherein the DNA is isolated from material shed from the infected host.

43. The method according to claim 42, material shed from the infected host is hair, fur, skin, exoskeleton, or a combination thereof.

44. The method according to claim 37, further comprising distinguishing the pathogen DNA from the infected host's DNA.

45. The method according to claim 26, wherein the sample is an RNA sample.

46. The method according to claim 26, wherein the sample is a cDNA sample.

47. The method according to claim 26, further comprising analyzing the target nucleic acid.

48. The method according to claim 47, wherein analyzing the target nucleic acid comprises sequencing all or a portion of the target nucleic acid.

49. A kit comprising:
a bridge oligonucleotide;
one or more splint oligonucleotides;
a rolling circle amplification primer;
a deoxynucleotide triphosphate (dNTP) mixture comprising modified nucleotides; and
instructions for using the components of the kit to produce target capture nucleic acids according to the method of claim 1.

50. A kit comprising:
the target capture nucleic acids of claim 25; and
instructions for using the target capture nucleic acids to capture a target nucleic acid.


SEQUENCE LISTING (25 sequences)

SEQ ID NO: 1 - DNA, 88 bp, Artificial sequence
  synthetic sequence
tttttttgattctaatttaaactattctctgttctttcatggggaagcagatttgggtaccacccaaggcgcgatcaagctttttttt

SEQ ID NO: 2 - DNA, 88 bp, Artificial sequence
  synthetic sequence
tttttttcatggggaagcagatttgggtaccacccaagtattgactcacccatcaacaaccgctatgtgcgcgatcaagctttttttt

SEQ ID NO: 3 - DNA, 88 bp, Artificial sequence
  synthetic sequence
tttttttgtattgactcacccatcaacaaccgctatgtatttcgtacattactgccagccaccatgaagcgcgatcaagctttttttt

SEQ ID NO: 4 - DNA, 88 bp, Artificial sequence
  synthetic sequence
ttttttttatttcgtacattactgccagccaccatgaatattgtacggtaccataaatacttgaccacgcgcgatcaagctttttttt

SEQ ID NO: 5 - DNA, 88 bp, Artificial sequence
  synthetic sequence
tttttttatattgtacggtaccataaatacttgaccacctgtagtacataaaaacccaatccacatcagcgcgatcaagctttttttt

SEQ ID NO: 6 - DNA, 88 bp, Artificial sequence
  synthetic sequence
tttttttcctgtagtacataaaaacccaatccacatcaaaaccccctccccatgcttacaagcaagtagcgcgatcaagctttttttt

SEQ ID NO: 7 - DNA, 88 bp, Artificial sequence
  synthetic sequence
tttttttaaaaccccctccccatgcttacaagcaagtacagcaatcaaccctcaactatcacacatcagcgcgatcaagctttttttt

SEQ ID NO: 8 - DNA, 88 bp, Artificial sequence
  synthetic sequence
tttttttacagcaatcaaccctcaactatcacacatcaactgcaactccaaagccacccctcacccacgcgcgatcaagctttttttt

SEQ ID NO: 9 - DNA, 88 bp, Artificial sequence
  synthetic sequence
tttttttaactgcaactccaaagccacccctcacccactaggataccaacaaacctacccacccttaagcgcgatcaagctttttttt

SEQ ID NO: 10 - DNA, 88 bp, Artificial sequence
  synthetic sequence
tttttttctaggataccaacaaacctacccacccttaacagtacatagtacataaagccatttaccgtgcgcgatcaagctttttttt

SEQ ID NO: 11 - DNA, 88 bp, Artificial sequence
  synthetic sequence
tttttttacagtacatagtacataaagccatttaccgtacatagcacattacagtcaaatcccttctcgcgcgatcaagctttttttt

SEQ ID NO: 12 - DNA, 88 bp, Artificial sequence
  synthetic sequence
ttttttttacatagcacattacagtcaaatcccttctcgtccccatggatgacccccctcagataggggcgcgatcaagctttttttt

SEQ ID NO: 13 - DNA, 88 bp, Artificial sequence
  synthetic sequence
tttttttcgtccccatggatgacccccctcagataggggtcccttgaccaccatcctccgtgaaatcagcgcgatcaagctttttttt

SEQ ID NO: 14 - DNA, 199 bp, Artificial sequence
  synthetic sequence
tttttttggtattttcgtctggggggtatgcacgcgatagcattgcgagacgcggcgcgcctttttttttagcaccctatgtcgcagtatctgtctttgattcctgcctcatcctattaggcgcgcctttttttttcctacgttcaatattacaggcgaacatacttactaaagtgtgttaattaggcgcgcctttttt

SEQ ID NO: 15 - DNA, 199 bp, Artificial sequence
  synthetic sequence
tttttgtaggacataataataacaattgaatgtctgcacagccactttccacaggcgcgccttttttttttaacaaaaaatttccaccaaaccccccctcccccgcttctggccacagcggcgcgcctttttttttcatctctgccaaaccccaaaaacaaagaaccctaacaccagcctaaccaggcgcgcctttttt

SEQ ID NO: 16 - DNA, 12 bp, Artificial sequence
  synthetic sequence
  misc_feature (1)..(1): The nucleotide at position 1 comprises 5AmMC6
  misc_feature (12)..(12): The nucleotide at position 12 comprises 3AmMO
aaaaaaaaaaaa

SEQ ID NO: 17 - DNA, 21 bp, Artificial sequence
  synthetic sequence
gcgcgatcaagcttttttttt

SEQ ID NO: 18 - DNA, 21 bp, Artificial sequence
  synthetic sequence
aaaaaaaaagcttgatcgcgc

SEQ ID NO: 19 - DNA, 17 bp, Artificial sequence
  synthetic sequence
ggcgcgccttttttttt

SEQ ID NO: 20 - DNA, 17 bp, Artificial sequence
  synthetic sequence
aaaaaaaaaggcgcgcc

SEQ ID NO: 21 - DNA, 33 bp, Artificial sequence
  synthetic sequence
gcgcgatcaagcttttttttttttttttttttt

SEQ ID NO: 22 - DNA, 20 bp, Artificial sequence
  synthetic sequence
  misc_feature (1)..(1): The nucleotide at position 1 comprises 5AmMC6
  misc_feature (13)..(20): n is a, c, g, or t
  misc_feature (20)..(20): The nucleotide at position 20 comprises 3AmMO
gcttgatcgcgcnnnnnnnn

SEQ ID NO: 23 - DNA, 19 bp, Artificial sequence
  synthetic sequence
  misc_feature (1)..(1): The nucleotide at position 1 comprises 5AmMC6
  misc_feature (1)..(8): n is a, c, g, or t
  misc_feature (19)..(19): The nucleotide at position 19 comprises 3AmMO
nnnnnnnnaaaaaaaaaaa

SEQ ID NO: 24 - DNA, 25 bp, Artificial sequence
  synthetic sequence
ggcgcgcctttttttttttttttgc

SEQ ID NO: 25 - DNA, 41 bp, Artificial sequence
  synthetic sequence
  misc_feature (1)..(1): The nucleotide at position 1 comprises 5AmMC6
  misc_feature (1)..(8): n is a, c, g, or t
  misc_feature (34)..(41): n is a, c, g, or t
  misc_feature (41)..(41): The nucleotide at position 1 comprises 3AmMO
nnnnnnnngcaaaaaaaaaaaaaaaggcgcgccnnnnnnnn